(19) United States
(12) Patent Application Publication
(10) Pub. No.: US 2024/0382211 A1
(43) Pub. Date: Nov. 21, 2024

(54) CLOTHING WITH BUILT IN TOURNIQUET FUNCTIONALITY

(71) Applicant: Elizardo ARISMENDEZ, Tucson, AZ (US)

(72) Inventor: Elizardo ARISMENDEZ, Tucson, AZ (US)

(21) Appl. No.: 18/665,425

(22) Filed: May 15, 2024

Related U.S. Application Data
(60) Provisional application No. 63/466,578, filed on May 15, 2023.

(51) Int. Cl. A61B 17/132; A41B 1/08; A41D 1/02; A41D 1/06

(52) U.S. Cl. CPC A61B 17/1327; A41B 1/08; A41D 1/02; A41D 1/06; A41B 2400/32; A41D 2400/32

(57) ABSTRACT

Disclosed embodiments relate to a tourniquet system which includes straps integrated into a pair of pants or shirt where the straps are configured to tighten to apply pressure to the leg or arm, respectively. The tourniquet system also includes fastening mechanisms which hold the straps in a tightened position and a rod to further tighten the strap and apply additional pressure to the leg or arm. The tourniquet system is configured to be used one handed and may be used with either a dominant or non-dominant hand. In some embodiments, the tourniquet system's straps are positioned around the thigh and the calf of each leg or the upper arm and forearm of the shirt to apply appropriate pressure.

### CROSS-REFERENCE TO RELATED APPLICATIONS

This application claims priority to and the benefit of U.S. Provisional Patent Application No. 63/466,578, filed on May 15, 2023, which is incorporated herein by reference in its entirety.

### BACKGROUND

Tourniquets have been used in trauma care to apply pressure to wounds to minimize or stop bleeding. Tourniquets have wide applications in emergency situations, surgery, post-operative rehabilitation, and in combat situations. Tourniquets in general can have a simplistic design only requiring an object like a stick or rod and an object to go around the area of the wound such as a rope or belt. While using a stick and rope can be used as a tourniquet, these makeshift tourniquets are ineffective compared to more advance devices.

Other tourniquet devices include silicone ring tourniquets, elastic ring tourniquets, combat application tourniquets, and surgical auto-transfusion tourniquets. While all of these specific tourniquets have specific applications, the tourniquet devices all are applied to the area post wound.

In extreme cases, such as combat, a wounded person may be in a dangerous situation or on their own where someone else cannot help apply a tourniquet to a wound such as a missing limb. In these cases, combat application tourniquets (CATs) come packaged as a prehospital tourniquet to be used on oneself. The package includes a strap that can be applied over a wound and then a buckle to hold the tourniquet in place to prevent bleeding. While this device can be effective in stopping bleeding, the device still requires the wounded person to either be able to apply the device on themselves or have someone else around to apply the device to the wound.

In sum, tourniquet devices can be effective when used correctly, however, certain circumstances make applying tourniquets difficult and sometimes impossible. In these cases, improper application or not applying a tourniquet can be a life or death situation. Accordingly, there is a long felt and ongoing need for efficient, low-cost, and high-performance ways to self-apply tourniquets where the tourniquet is still effective in reducing or stopping blood flow.

### BRIEF SUMMARY

The present disclosure relates to a tourniquet system and methods of using the tourniquet system. Embodiments described herein solve one or more problems related to effectively self-applying a tourniquet system easily and efficiently. More particularly, embodiments of the invention may have applications in combat circumstances where soldier wounds require instant care using a tourniquet when the solider is injured and either alone or with others who cannot help.

Embodiments of the tourniquet system include a pair of pants with straps integrated in the pant legs. Some embodiments have one strap while other embodiments may have multiple straps. The straps may be made of a nylon material. The straps may be placed in strategic areas along the legs of the pants where wounds requiring tourniquets most often occur. Some examples may include around the upper thigh and/or calf.

Embodiments of the tourniquet system include a shirt with straps integrated in the shirt arms. Some embodiments have one strap while other embodiments may have multiple straps. The straps may be made of a nylon material. The straps may be placed in strategic areas along the arms of the shirt where wounds requiring tourniquets most often occur. Some examples may include around the upper arm and forearm.

Embodiments of the invention further include one or more locking mechanisms such as a ladder locks. The locking mechanism may be included on both sides of each strap to allow the tourniquet to be tightened by either a dominant or non-dominant hand. Some embodiments additionally include a rod (often referred to as a windlass in the context of tourniquets) to enable twisting of the strap to add additional pressure to the underlying anatomy.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B illustrates an example of a tourniquet system integrated into a pair of pants showing close ups of a strap and a rod;

FIG. 1C illustrates an example rod that may be included in the tourniquet system, the strap passing through the rod so that the rod can be used to twist and thereby tighten the strap;

FIGS. 2A and 2B illustrate another example of a tourniquet system integrated into a pair of pants;

FIG. 2C illustrates an example of a strap with a ladder lock on each end and an additional strap piece for connecting to the ladder locks and extending therebetween;

FIGS. 3-5 illustrate an example of the tourniquet system integrated into a shirt, with FIG. 3 providing a front view, FIG. 4 providing a rear view, and FIG. 5 providing a front view of the shirt in a different configuration; and

FIG. 6 illustrates an example of a tourniquet system integrated into a pair of pants including a loop securing mechanism.

### DETAILED DESCRIPTION

### Example Embodiments

The following detailed description solves one or more of the problems discussed above. As explained in greater detail below, the tourniquet system may be used to self-apply a tourniquet to a leg wound. One example is in combat where a leg and arm may be missing and a tourniquet must be applied to the missing leg with only one arm. Other applicable scenarios may include bleeding from a bullet wound in the leg requiring a tourniquet to stop bleeding.

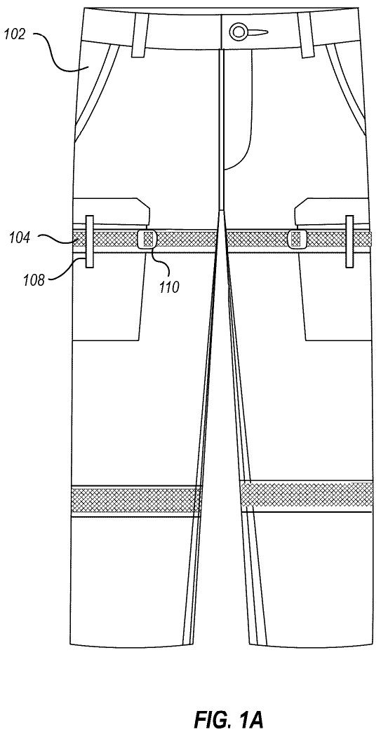

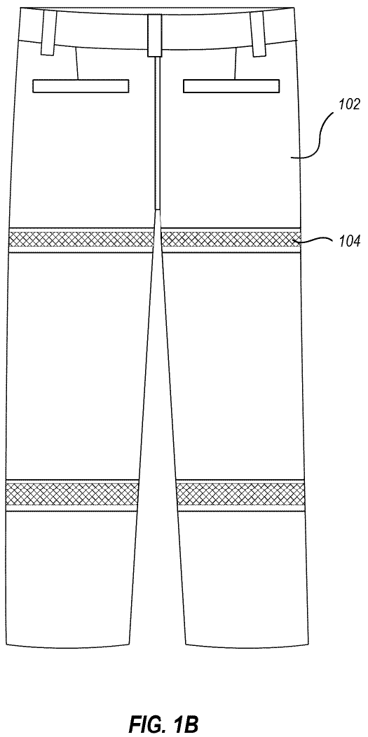

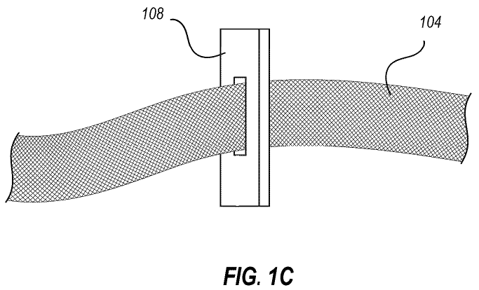

FIGS. 1A through and 2C illustrate an example of a tourniquet system integrated into a pair of pants. The tourniquet system includes a pair of pants 102 which may be, for example, a pair of military style pants. The pair of pants include a strap 104 which goes around the circumference of a leg of the pants 102. The tourniquet system may include one or more straps 104. For example, FIGS. 1A and 1B illustrates four straps. The straps 104 may be strategically positioned on both legs of the pants 102 and along the legs of the pants 102. For example, the straps 104 may be positioned to go around the upper thigh (e.g., the upper half of the thigh) and the calf of each leg of the pants. The upper thigh and calf may be chosen because the thigh and calf are common areas that require tourniquets when a leg injury occurs. Other areas such as above the knee, below the knee, or at the ankle may alternatively be chosen for placement of the straps or for additional straps.

The strap 104 is favorably made of a material which when tightened applies adequate pressure to minimize and/or stop bleeding. Examples of materials from which the strap 104 may be formed include nylon, polyester, polypropylene, and/or other materials with sufficient structure and strength to apply sufficient pressure when tightened. The length of the strap 104 should be long enough to go around the leg of the pants 102. The width of the strap 104 may vary to allow the strap to apply enough pressure to minimize or stop bleeding. Examples of appropriate widths of the strap 104 include about 3 inches, about 2.5 inches, about 2 inches, about 1.75 inches, about 1.5 inches, about 1 inch, or a width between any of the foregoing values. The length and width of each strap 104 may vary based on where the strap is located. For example, the length of a strap around the thigh may be longer than the length of a strap around the calf. Additionally, more pressure may be needed around the thigh than the calf therefore resulting in a wider strap around the thigh and a narrower strap around the calf.

The strap 104 may be incorporated into the pants 102 in a variety of ways. For example, the straps 104 may be incorporated into the inseams of the pants 102. The straps 104 may be attached to the pants 102 during manufacture by stitching the straps into the pants or attaching them by other means such as pins, rivets, staples, adhesive, zippers, and/or other fastening mechanisms suitable for clothing. The straps 104 alternatively may be incorporated into the pants 102 by being incorporated into a pocket like structure. The pants 102 may include a partial like pocket structure where straps 104 may be slide into during or after the pants 102 have been produced. In some embodiments, the straps 104 may be fully exposed while other embodiments include a pocket that covers an otherwise exposed portion of the straps 104.

Some embodiments additionally include a rod 108 (shown in detail in FIG. 1C) which can be used to apply additional pressure to the tourniquet by enabling twisting of the strap 104. The strap 104 is passed through a slit formed in the rod 108. When the rod 108 is rotated end over end, the strap 104 is twisted with it and tightens accordingly. Other embodiments may include different rods such as solid rods affixed to or embedded into the strap, clip on rods which are selectively attachable to the strap, or other appropriate means to attach the rod 108 to the strap 104. The rod 108 may be formed of plastic, metal, or other material suitable for allowing the rod 108 to further tighten the strap 104.

In the configuration shown in FIG. 1A, the rod 108 is in a vertical position. Other embodiments can orient the rod 108 by default in a horizontal position (or some non-vertical position), with sufficient slack in the strap 104 to accommodate the slight twist from such orientation. The rod 108 can be positioned along an edge of the pocket and/or be disposed within the pocket to keep it out of the way unless/until needed. The slot of the rod 108 can be positioned in the center of the length of the rod 108. Alternatively, the slot can be offset from the center such that one end of the rod is longer than the other. For example, if the strap 104 is located near a pocket opening, one end of the rod 108 may be shorter than the other to minimize interference with the pocket (e.g., to avoid sticking up out of the pocket and/or avoid interfering with pocket closure) while the longer end can extend farther into the pocket.

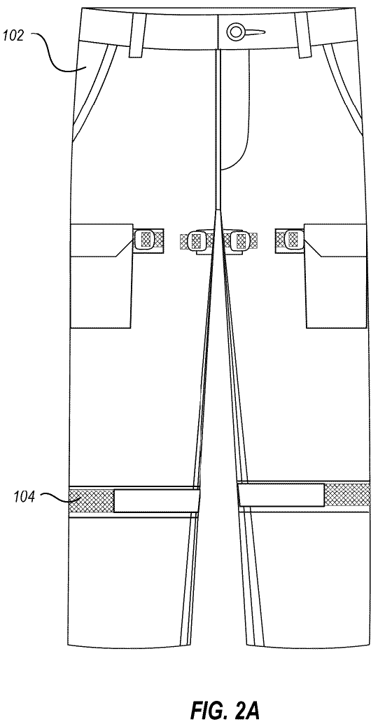

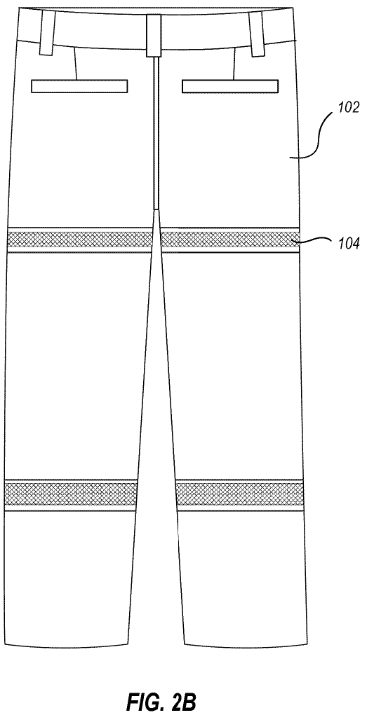

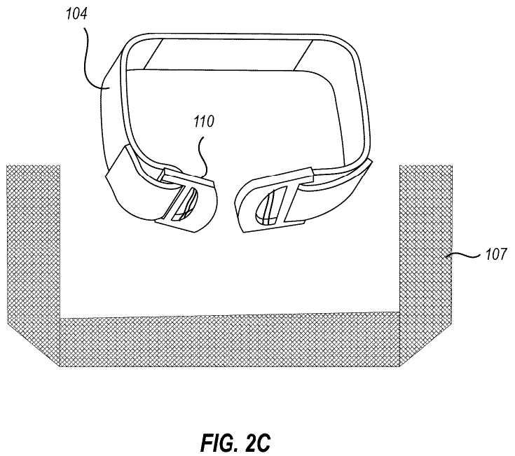

FIGS. 2A and 2B show a configuration where at least a portion of the straps 104 are covered by pockets found on the legs of the pants 102.

The tourniquet system 100 also includes a fastening mechanism that holds the straps 104 in place when the straps 104 and tightened. An example of a fastening mechanism shown in the illustrated embodiments is a ladder lock 110. In other embodiments, the fastening mechanism may be a puzzle piece lock, a friction lock, a snap lock, a knotting mechanism, a pin locking structure, or other appropriate structures configured to allow tightening of the strap and to keep the strap 104 in a tightened position. It will be understood that examples described herein as including ladder locks 110 can additionally or alternatively include other such fastening mechanisms.

Some embodiments, such as shown in FIGS. 1A and 1B, only include one ladder lock 110 per strap 104. For example, the free end of the strap opposite the end with the ladder lock 110 can wrap around the leg and be passed through the ladder lock 110. Further pulling of the free end enables tightening of the strap 104.

Other embodiments, such as shown in FIGS. 2A and 2B, include multiple ladder locks 110 per strap 104. For example, the strap 104 may include a ladder lock 110 on each side of the strap 104 as shown in detail in FIG. 2C, with an additional strap piece 107 included to close the gap between the ladder locks 110. The additional strap piece 107 can includes ends that are passed through respective ladder locks 110 of the strap 104. In this embodiment, the person wearing the pants may pull on either end of the strap piece 107 and engage one of the respective ladder locks 110. This beneficially allows use of either hand (e.g., dominant or non-dominant hand) to tighten the tourniquet.

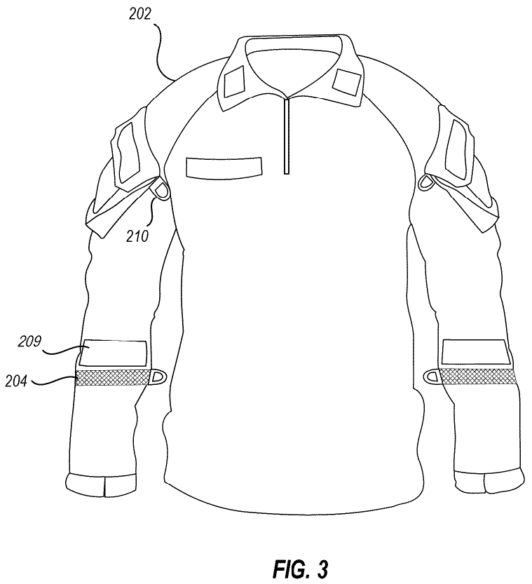

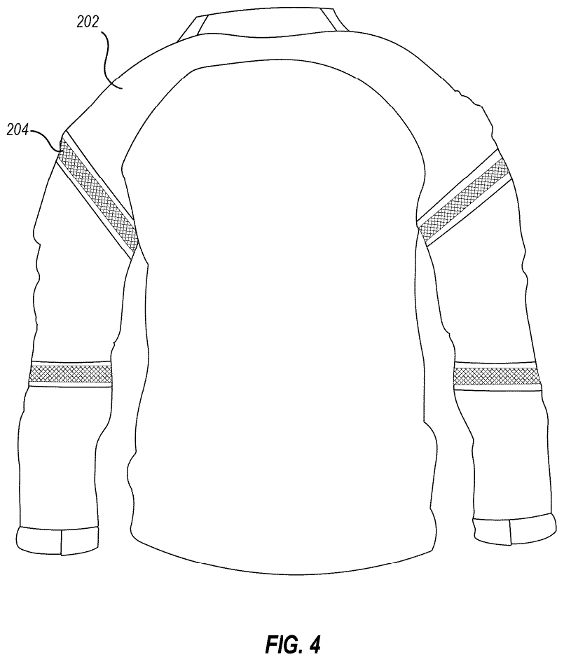

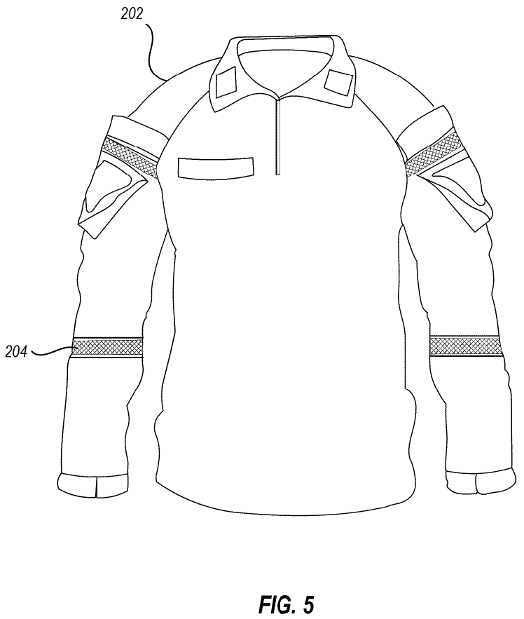

FIGS. 3-5 illustrate an example of a tourniquet system integrated into a shirt as a front view and a back view, respectively. Principles and components discussed above relative to the tourniquet system integrated into pants 102 are also applicable to the below discussion.

The tourniquet system includes a shirt 202 which may be, for example, a military shirt. The shirt includes a strap 204 which goes around the circumference of an arm of the shirt 202. The tourniquet system may include one or more straps 204. For example, the shirt 204 may include four straps. The straps 204 may be strategically positioned on both arms of the shirt 202. For example, the straps 204 may be positioned to go around the upper arm and the forearm of each arm of the shirt 202. The upper arm and forearm may be chosen due to the fact these portions of the arms are common areas that require tourniquets when an arm injury occurs. Other areas such as above the elbow, mid-arm, or at the wrist may alternatively be chosen for placement of the straps or for additional straps.

The straps 204 may be incorporated into the shirt 202 in a variety of ways. The straps 204 may be attached to the shirt 202 production by stitching the straps 204 into the shirt or attaching by other means such as pins or staples. The straps 204 alternatively may be incorporated into the shirt 202 by being surrounded by fabric on both sides by a pocket like structure. The shirt 202 may include a partial like pocket structure where straps 204 may be slide into during or after the shirt 202 has been produced. In some embodiments, the straps 204 may be fully exposed. Other embodiments can include pockets and/or flaps (e.g., flap 209 and shoulder pockets) that cover an otherwise exposed portion of the straps 204.

As discussed above, the straps 204 can be made of a material which when tightened has sufficient integrity to apply adequate pressure to minimize and/or stop bleeding. Examples of materials the strap 204 may be formed of include nylon, polyester, polypropylene fabric, or other materials which apply enough pressure when tightened. The length of the strap 204 should be long enough to go around the arm of the shirt 202. The width of the strap 204 may vary to allow the strap to apply enough pressure to minimize or stop bleeding. Examples of appropriate widths of the strap 204 may include about 3 inches, about 2.5 inches, about 2 inches, about 1.75 inches, about 1.5 inches, about 1 inch, or a width between the above forementioned values. The length and width of each strap 204 may vary based on where the strap is located. For example, the length of a strap around the upper arm may be longer than the length of a strap around the forearm. Additionally, more pressure may be needed around the upper arm than the forearm therefore resulting in a wider strap.

The tourniquet system can include locks, such as ladder locks 210 and/or any other locking means disclosed herein. Each strap 204 can be associated with a rod to assist in twisting and further tightening the strap 204, as discussed above. Where rods are included, they may be oriented within or against adjacent pockets or flaps to hide the rod from normal view and/or to keep it conveniently oriented.

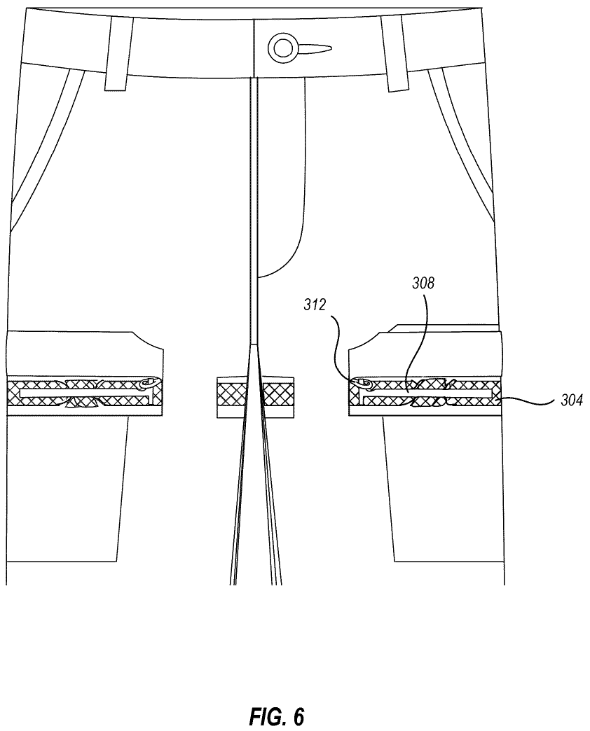

FIG. 6 illustrates an example tourniquet system 300 integrated into a pair of pants and including a securing mechanism for securing a rod 308 and preventing it from spinning and untwisting/loosening the strap 304. The securing mechanism may be included in any of the other embodiments disclosed herein and can be associated with one or more of the straps of the article of clothing.

FIG. 6 shows an example loop 312 that can be affixed to the article of clothing (the pants in this example) and positioned near any of the straps 304. The loop 312 may be formed of an elastic material, bungee material, metal material, or other appropriate materials. The loop 312 may be pre-closed or may be selectively openable. For example, the loop 312 may include two opposing strands with hook and loop fastener material (and/or other fastening mechanisms) such that the opposing strands can be selectively attached to form the closed loop 312 and selectively detached to open the loop 312.

In some embodiments the loop 312 may be stitched into the top corner of a pocket. In other embodiments, the loop 312 may be stitched into another area of the pocket. In yet other embodiments, the loop 312 may be stitched into the pair of pants or shirt in locations without a pocket.

The loop 312 can be coupled to the rod 308 when the rod 308 has been twisted to tighten the strap 304. When the rod 308 is rotated end over end to twist and tighten the associated strap 304, the twisted strap 304 will tend to unwind (spinning the rod 308 with it) unless held in place. The loop 312 can be attached to the rod 308 to prevent the rod 308 from spinning and thereby prevent the strap 304 from loosening.

In embodiments where the strap 304 and rod 308 are located near flap, such as near a pocket with a flap, the flap can be closed over the rod 308 and fastened (e.g., using hook and loop fastener materials) to secure the rod 308 against unwanted rotation.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “strap”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A tourniquet system, comprising:
an article of clothing, the article of clothing comprising limb portions;
a first strap integrated into a first limb portion, wherein the first strap is substantially parallel to a circumference that goes around the first limb portion;
a first fastening mechanism associated with the first strap, the first fastening mechanism configured to hold the first strap in a closed circular configuration and to prevent loosening of the first strap; and
a rod associated with the first strap, the first strap passing through a slit within the rod, the rod being rotatable to enable twisting of the first strap.

2. The tourniquet system of claim 1, wherein the first fastening mechanism comprises a ladder lock.

3. The tourniquet system of claim 1, wherein the first strap has a width of about 1.5 inches to about 2.5 inches.

4. The tourniquet system of claim 1, wherein the rod is positioned, in an untightened state, alongside a pocket or within a pocket of the article of clothing.

5. The tourniquet system of claim 1, further comprising a loop affixed to the article of clothing, wherein the loop is positioned to engage with the rod to secure the rod against rotation.

6. The tourniquet system of claim 5, wherein the loop is positioned underneath or adjacent to a flap of the article of clothing, wherein the flap is closable over the rod to aid in securing the rod against rotation.

7. The tourniquet system of claim 5, wherein the loop is permanently closed.

8. The tourniquet system of claim 5, wherein the loop is selectively openable and closable.

9. The tourniquet system of claim 1, further comprising:
a second strap integrated into a second limb portion, wherein the second strap is substantially parallel to a circumference that goes around the second limb portion; and
a second fastening mechanism associated with the second strap, the second fastening mechanism configured to hold the second strap in a closed circular configuration and to prevent loosening of the second strap.

10. The tourniquet system of claim 9, wherein the article of clothing is pants.

11. The tourniquet system of claim 10, further comprising:
a third strap integrated into a first leg of the pants, wherein the third strap is substantially parallel to a circumference that goes around the leg;
a third fastening mechanism configured to hold the third strap in a closed circular configuration and to prevent loosening of the third strap;
a fourth strap integrated into a second leg of the pants, wherein the fourth strap is substantially parallel to a circumference that goes around the second leg;
a fourth fastening mechanism configured to hold the fourth strap in a closed circular configuration and to prevent loosening of the fourth strap.

12. The tourniquet system of claim 11, wherein the first strap is positioned around a thigh portion of the first leg of the pants, and the second strap is positioned around a thigh portion of a second leg of the pants.

13. The tourniquet system of claim 12, wherein the third strap is positioned below a knee portion of the first leg of the pants, and the fourth strap is positioned below a knee portion of the second leg of the pants.

14. The tourniquet system of claim 9, wherein the article of clothing is a shirt or jacket.

15. The tourniquet system of claim 14, further comprising:
a third strap integrated into a first arm of the shirt or jacket, wherein the third strap is substantially parallel to a circumference that goes around the arm;
a third fastening mechanism configured to hold the third strap in a closed circular configuration and to prevent loosening of the third strap;
a fourth strap integrated into a second arm of the shirt or jacket, wherein the fourth strap is substantially parallel to a circumference that goes around the second arm;
a fourth fastening mechanism configured to hold the fourth strap in a closed circular configuration and to prevent loosening of the fourth strap.

16. The tourniquet system of claim 15, wherein the first strap is positioned around an upper arm portion of the first arm of the shirt and the second strap is positioned around an upper arm portion of the second arm of the shirt.

17. The tourniquet system of claim 16, wherein the third strap is positioned around a forearm portion of the first arm of the shirt and the fourth strap is positioned around a forearm portion of the second arm of the shirt.

18. The tourniquet system of claim 1, wherein the first strap is associated with two fastening mechanisms.

19. A tourniquet system, comprising:
an article of clothing, the article of clothing comprising limb portions;
a first strap integrated into a first limb portion, wherein the first strap is substantially parallel to a circumference that goes around the first limb portion;
a first fastening mechanism associated with the first strap and configured as a ladder lock, the first fastening mechanism configured to hold the first strap in a closed circular configuration and to prevent loosening of the first strap;
a rod associated with the first strap, the first strap passing through a slit within the rod, the rod being rotatable to enable twisting of the first strap, wherein the rod is positioned, in an untightened state, alongside a pocket or within a pocket of the article of clothing; and
a loop affixed to the article of clothing, wherein the loop is positioned to engage with the rod to secure the rod against rotation, wherein the loop is positioned underneath or adjacent to a flap of the article of clothing, wherein the flap is closable over the rod to aid in securing the rod against rotation.

20. The tourniquet system of claim 19, wherein the article of clothing is pants, a shirt, or a jacket.
